(19) United States
(12) Patent Application Publication
(10) Pub. No.: US 2014/0171908 A1
(43) Pub. Date: Jun. 19, 2014

(54) Intra-Myocardial Agent Delivery Device, System and Method

(71) Applicant: Robert G Matheny, Norcross, GA (US)

(72) Inventor: Robert G Matheny, Norcross, GA (US)

(21) Appl. No.: 14/093,424

(22) Filed: Nov. 30, 2013

Related U.S. Application Data
(60) Provisional application No. 61/738,294, filed on Dec. 17, 2012.

(51) Int. Cl. A61M 5/14

(52) U.S. Cl. CPC A61M 5/14
USPC 604/508; 604/173; 604/151

(57) ABSTRACT

An intra-myocardial agent delivery device comprising a myocardial tissue access line that is configured to receive a pharmacological agent therein and at least a first agent delivery line having a central lumen therethrough, the first agent delivery line being in fluid communication with the tissue access line and including a plurality of dispersal lumens extending through the delivery line wall, wherein, when the first agent delivery line is disposed in myocardial tissue and the pharmacological agent is introduced into the tissue access line, the plurality of dispersal lumens allow the pharmacological agent to be transferred from the central lumen to the myocardial tissue.

### CROSS-REFERENCE TO RELATED APPLICATIONS

This application claims the benefit of U.S. Application No. 61/738,294, filed on Dec. 17, 2012.

### FIELD OF THE INVENTION

The present invention relates to systems and methods for delivering pharmacological agents to biological tissue. More particularly, the present invention relates to devices, systems and methods for site specific delivery of pharmacological agents and compositions to damaged and diseased cardiovascular tissue; particularly, myocardial tissue, and means for implanting and using the delivery systems to enable delivery of pharmacological agents and compositions to cardiovascular tissue.

### BACKGROUND OF THE INVENTION

### Anatomy of the Heart

The heart is surrounded by the pericardium, which is a sac consisting of two layers of tissue (fibrous pericardium and parietal layer of the serous pericardium). The pericardial space (between the pericardium and the heart) contains some pericardial fluid that bathes the outer tissue heart in a stable osmotic and electrolytic environment.

The heart tissue itself consists of four layers; the visceral layer of the serous pericardium, an adipose layer containing embedded arteries and veins, the myocardium, which is the major, muscular layer of the heart, and the inner epithelial layer, called the endocardium.

The coronary arteries are the first vessels to branch off the aorta. Through these arteries, the heart receives (at rest) about 5% of the cardiac output. Coronary blood flow is governed by a pressure gradient and by resistance of the vessels.

### Ischemic Disease of the Heart

Myocardial infarction is a common presentation of ischemic heart disease/coronary artery disease. The World Health Organization estimated in 2004 that 12.2% of worldwide deaths occurred as a result of ischemic heart disease. Ischemic heart disease was also deemed the leading cause of death in middle to high income countries and second only to respiratory infections in lower income countries. The Global Burden of Disease: World Health Organization 2004 Update, Geneva (2008). Worldwide more than 3 million people present with a ST elevation myocardial infarction (STEMI) and 4 million people present with a non-ST elevation myocardial infarction (NSTEMI) a year. White, et al., Acute Myocardial infarction, Lancet 372 (9638), pp. 570-84 (August 2008).

Rates of death from ischemic heart disease have slowed or declined in most high income countries, although cardiovascular disease still accounted for 1 in 3 of all deaths in the USA in 2008. Roger, et al., Executive summary: Heart Disease and Stroke Statistics—2012 update: A report from the American Heart Association, Circulation 125 (1), pp. 188-97 (January 2012).

In contrast, ischemic heart disease is becoming a more common cause of death in the developing world. For example in India, ischemic heart disease had become the leading cause of death by 2004; accounting for 1.46 million deaths (14% of total deaths). Deaths in India due to ischemic heart disease were also expected to double during 1985-2015. Gupta, et al., Epidemiology and Causation of Coronary Heart Disease and Stroke in India, Heart 94 (1), pp. 16-26 (January 2008).

Globally, it is predicted that disability adjusted life years (DALYs) lost to ischemic heart disease will account for 5.5% of total DALYs in 2030, making it the second most important cause of disability (after unipolar depressive disorder), as well as the leading cause of death by this date.

A myocardial infarction (a common presentation of ischemic heart disease) often occurs when a coronary artery becomes occluded and can no longer supply blood to the myocardial tissue. The consequences of a myocardial infarction are often severe and disabling. When a myocardial infarction occurs, the myocardial tissue that is no longer receiving adequate blood flow dies and is replaced with scar tissue. The infarct (or infracted) tissue cannot contract during systole and can actually undergo lengthening in systole, resulting in immediate depression in ventricular function. The abnormal motion of the infarct tissue can cause delayed or abnormal conduction of electrical activity to the still surviving peri-infarct tissue (tissue at the junction between the normal tissue and the infarcted tissue) and also places extra structural stress on the peri-infarct tissue.

Thus, in addition to immediate hemodynamic effects, the infarct tissue and the myocardium tissue undergo three major processes: infarct expansion, infarct extension, and chamber remodeling. These factors, individually and in combination, contribute to the eventual dysfunction observed in the cardiovascular tissue remote from the site of the infarction.

Infarct expansion is a fixed, permanent, disproportionate regional thinning and dilatation of tissue within the infarct zone. Infarct expansion occurs early after a myocardial infarction. The mechanism of infarct expansion is slippage of the tissue layers.

Infarct extension is additional myocardial necrosis following myocardial infarction. Infarct extension results in an increase in total mass of infarct tissue. The additional infarct tissue can also undergo infarct expansion.

Infarct extension occurs days after a myocardial infarction. The mechanism for infarct extension is believed to be an imbalance in the blood supply to the peri-infarct tissue versus the increased oxygen demands on the tissue.

Remodeling is usually the progressive enlargement of the ventricle accompanied by a depression of ventricular function. Myocyte function in the cardiac tissue remote from the initial myocardial infarction becomes depressed. Remodeling occurs weeks to years after myocardial infarction.

Such remodeling usually occurs on the left side of the heart. Where remodeling does occur on the right side of the heart, it can generally be linked to remodeling (or some other negative event) on the left side of the heart. Remodeling can occur independently in the right heart, albeit less often than the left.

There are many potential mechanisms for remodeling, but it is generally believed that the high stress on peri-infarct tissue plays an important role. Due to a variety of factors, such as altered geometry, wall stresses are much higher than normal in the cardiovascular tissue surrounding the infarction.

The processes associated with infarct expansion and remodeling are believed to be the result of high stresses exerted at the junction between the infarct tissue and the normal cardiovascular tissue (i.e., the peri-infarct region). In the absence of intervention, these high stresses will eventually kill or severely depress cell function in adjacent cells. As a result, the peri-infarct region will therefore grow outwardly from the original infarct site over time. This resulting wave of dysfunctional tissue spreading out from the original myocardial infarct region greatly exacerbates the nature of the disease and can often progress into advanced stages of heart failure.

Various methods for treating a myocardial infarction are often employed. Such methods include stabilizing the hemodynamics associated with a myocardial infarction via systemic delivery of various pharmacological agents and restoring the patency of occluded vessels via thrombolytic therapy or angioplasty and stents.

Several additional methods for treating a myocardial infarction are directed to re-establishing blood flow to the ischemic area through stimulation of angiogenesis. Re-establishing blood flow at the ischemic area can, and in many instances will, reduce symptoms associated with a myocardial infarction and/or improve cardiac function.

Some methods for re-establishing blood flow and rehabilitating the heart involve invasive surgery, such as bypass surgery or angioplasty. Other methods employ lasers to bore holes through the infarctions and ischemic area(s) to promote blood flow. As one can readily appreciate, there are numerous incumbent risks associated with the noted methods.

A further method for treating a myocardial infarction is the “direct” or selective delivery of pharmacological agents to the infarction and/or ischemic area (i.e. effected or damaged cardiovascular tissue).

Various surgical approaches have also been employed to treat a myocardial infarction, including approaches to exclude, isolate, or remove the infarct region, and surround the heart, or a significant portion thereof, with a jacket or mesh type prosthesis to prevent remodeling.

The conventional methods and systems for treating damaged or diseased cardiovascular structures and tissue, including infarct tissue, are discussed in detail below.

### Direct Delivery of Pharmacological Agents to Cardiovascular Tissue

As indicated above, one commonly employed method of treating a myocardial infarction is the direct or selective delivery of pharmacological agents to the infarction and/or ischemic area. Direct delivery of a pharmacological agent to the effected cardiovascular tissue is often preferred over the systemic delivery for several reasons. A primary reason is that a substantially greater concentration of such agents that can be delivered directly into the effected cardiovascular tissue, compared with the dilute concentrations possible through systemic delivery. Another reason is the risk of systemic toxicity which can, and in many instances will, occur with doses of pharmacological agents that are typically required to achieve desired drug concentrations in the effected cardiovascular tissue.

One common method of delivering pharmacological agents directly to effected or damaged myocardial tissue, e.g., infarct region, comprises advancing a catheter through the vasculature and into the heart to inject the agents directly into the effected cardiovascular tissue from within the heart.

Another method of delivering pharmacological agents directly to effected cardiovascular tissue comprises epicardial injection into the tissue during an open chest procedure. The agents that can be, and have been, administered to the effected cardiovascular tissue include various pharmacological agents, such as antithrombotic agents, e.g., heparin, hirudin, and ticlopidine.

Various controlled release agent delivery methods and systems have also been disclosed. Folkman, et al. in Drug Pacemakers in the Treatment of Heart Block, New York Acad. Sci., p. 857 (Jun. 11, 1964) describe a wax or silicone rubber capsule that is capable of being filled with candidate active agents. In open chest animal studies, the capsule was tunneled into the epicardial tissue. After being thus positioned, the capsule released the agent(s) producing quantifiable effects on heart rate for four to five days.

Labhasetwar, et al. in Epicardial Administration of Ibutilide rom Polyurethane Matrices: Effects on Defibrillation Threshold and Electrophysiologic Parameters, J. Cardiovasc. Pharm., vol. 24, pp. 826-840 (1994,) describe the reduction of defibrillation thresholds using a epicardially positioned patch containing Ibutilide in an acute canine model.

U.S. Pat. No. 5,154,182 also describes an implantable patch electrode that is surgically attached to the epicardium and capable of delivering a pharmacological agent.

Various other methods and devices have been developed for delivering pharmacological agents directly to cardiovascular tissue. Illustrative are the myocardial implants disclosed in U.S. Pat. Nos. 6,258,119 and 6,053,924.

In U.S. Pat. No. 6,258,119 a myocardial implant for insertion into a heart wall for trans myocardial revascularization (TMR) of the heart wall is disclosed. The implant provides a means to promote angiogenesis, and has a flexible, elongated body that contains a cavity and openings through the flexible, elongated body from the cavity. The TMR implant includes a coaxial anchoring element integrally formed at one end for securing the TMR implant in the heart wall.

U.S. Pat. No. 6,053,924 also describes a medical device for performing TMR in a human heart. The device consists of a myocardial implant and a directable intracardiac catheter for delivery into a heart wall.

Various mechanical means have also been employed to delivery one or more pharmacological agents directly to cardiovascular tissue. In U.S. Pat. No. 5,551,427 an implantable helical injection needle, which can be screwed into the heart wall and connected to an implanted drug reservoir outside the heart, is disclosed. The implantable system facilitates the delivery of pharmacological agents directly into the wall of the heart acutely by injection from the proximal end, or on an ongoing basis by a proximally located implantable subcutaneous port reservoir, or pumping mechanism.

The agent delivery can be performed by a number of techniques, among them infusion through a fluid pathway, and delivery from controlled release matrices at a depth within the heart. Controlled release matrices are described as drug polymer composites in which a pharmacological agent is dispersed throughout a pharmacologically inert polymer substrate.

U.S. Pat. No. 8,027,740 similarly discloses an implantable helical injection needle, which can be screwed into the heart wall and connected to an implanted drug reservoir outside the heart.

U.S. Pat. No. 6,971,998 discloses an implantable drug-carrying coil or dart, which can be inserted into the center of the myocardium and isolated from the internal chambers of the heart and pericardial space outside the heart. The coil or dart is positioned in the myocardium via a catheter that is navigated through the patient's arteries.

### Surgical Treatments

As also indicated above, various surgical approaches have been employed to treat a myocardial infarction. The surgical approaches include various means to exclude, isolate, or remove the infarct region.

One surgical approach that has been employed is to apply heat to the infarct region to shrink the infarcted tissue, followed by suturing a patch onto the infarct region.

Another surgical approach is to surround the heart, or a significant portion thereof, with a jacket or mesh type prosthesis to prevent remodeling. Illustrative are the prostheses disclosed in U.S. Pat. Nos. 6,508,756 and 5,800,528.

A further surgical approach is to form a reinforcement region proximate infarct tissue. Illustrative is the method disclosed in U.S. Pub. No. 2012/0059355, wherein a reinforcement region is formed within the myocardium by introducing a delivery device through a vessel wall and delivering a biomaterial, e.g., fibrin glue, to a desired treatment site, i.e. infarct tissue or tissue within a border region adjacent to the infarct tissue.

There are various drawbacks and disadvantages associated with the noted methods for treating damaged or diseased cardiovascular structures and tissue; particularly, infarct tissue. A major drawback associated with many, if not all, of the agent delivery systems and methods is that they are devoid of effective means for assuring that a precise dose of a pharmacological agent is delivered to the treatment site.

A further drawback is that most of the implantable delivery devices include lumens or other components that are constructed from various polymeric materials, such as poly(ethylene terephthalate) (PET). Such components can, and often will, cause irritation and undesirable biologic responses from surrounding biological tissue(s).

A major drawback associated with surgical approaches for treating damaged or diseased cardiovascular structures and tissue is that the surgical techniques typically require a highly-invasive open chest procedure to access the heart. Such a procedure often poses the risk of infection and carries additional complications, such as instability of the sternum, post-operative bleeding, and mediastinal infection. The thoracic muscle and ribs are also severely traumatized, and the healing process results in an unattractive scar. Post-operatively, most patients endure significant pain and must forego work or strenuous activity for a long recovery period.

It would thus be desirable to provide effective and accurate means for delivering pharmacological agents directly to cardiovascular tissue to treat a cardiovascular disorder and/or damaged or diseased cardiovascular tissue; particularly, infarct tissue, that substantially reduces or eliminates the noted drawbacks and disadvantages associated with existing means for delivering pharmacological agents to cardiovascular tissue.

It would also be desirable to provide effecting means for reinforcing the myocardium or a refract region thereof that does not require a highly-invasive open chest procedure.

It is therefore an object of the present invention to provide intra-myocardial agent delivery systems and methods that provide effective and accurate means for delivering pharmacological agents directly to cardiovascular tissue and/or regions proximate thereto to treat damaged or diseased cardiovascular tissue, such as infarct tissue, that substantially reduce or eliminate the drawbacks and disadvantages associated with existing means for delivering pharmacological agents to cardiovascular tissue.

It is another object of the present invention to provide intra-myocardial agent delivery systems and methods that can be employed to reinforce the myocardium or a refract region thereof and do not require an open chest procedure for placement proximate the myocardium.

It is another object of the present invention to provide extracellular matrix (ECM) compositions, which, when delivered to damaged biological tissue; particularly, cardiovascular tissue, induce neovascularization, host tissue proliferation, bioremodeling, and regeneration of cardiovascular tissue and associated structures with site-specific structural and functional properties.

These and other objects, advantages, and features of the invention will become apparent to those persons skilled in the art upon reading the details of the invention as more fully described below.

### SUMMARY OF THE INVENTION

The present invention is directed to intra-myocardial agent delivery devices, systems and methods for site specific delivery of pharmacological agents and compositions to damaged and diseased cardiovascular tissue; particularly, myocardial tissue, and means for implanting and using the delivery devices and systems to enable delivery of pharmacological agents and compositions to cardiovascular tissue.

In some embodiments of the invention, the intra-myocardial agent delivery devices also comprise myocardium reinforcing members.

Cardiovascular conditions that are amenable to treatment according to the invention include any pathological condition that is amenable to treatment by increasing the number of functional coronary blood vessels, including, without limitation, ischemic heart disease; particularly, myocardial infarction, arrhythmia, cardio-myopathy, coronary angioplasty restenosis, atherosclerosis of a coronary artery, thrombosis, a cardiac condition related to hypertension, cardiac tamponade, and pericardial effusion.

In a preferred embodiment of the invention, the intra-myocardial agent delivery devices include a central tube and at least one, more preferably, a plurality of agent delivery tubes that are in fluid communication with the central tube.

According to the invention, the intra-myocardial agent delivery devices of the invention can include any number of agent delivery tubes to, for example, deliver pharmacological agents to the desired number of delivery sites and/or provide the desired degree of reinforcement for the myocardium.

In a preferred embodiment of the invention, the central tube includes a central lumen that extends through the central tube and is configured to receive and transfer a pharmacological agent or composition therethrough. Each agent delivery tube also includes a central lumen that is similarly configured to facilitate the transfer of a pharmacological agent or composition therethrough.

In a preferred embodiment of the invention, at least one, more preferably, each agent delivery tube has at least one, more preferably, a plurality of laterally positioned perforations or lumens that facilitate delivery of pharmacological agents and compositions from the delivery tubes to adjacent biological tissue.

In a preferred embodiment, the agent delivery devices are adapted to delivery at least one pharmacological agent or composition to a biological tissue site, e.g. infarct region, which can comprise, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises an angiogenic factor, growth factor, antihypertensive agent, inotropic agent, antiatherogenic agent, beta-blocker, sympathomimetic agent, phosphodiesterase inhibitor, diuretic, vasodilator, thrombolytic agent, cardiac glycoside, and/or antineoplastic agent.

In some embodiments of the invention, the pharmacological agent comprises at least one Class I, II, III or IV anti-arrhythmic agent.

In some embodiments of the invention, the pharmacological agent comprises a statin. According to the invention, suitable statins include, without limitation, atorvastatin, cerivastatin, fluvastatin, lovastatin, mevastatin, pitavastatin, pravastatin, rosuvastatin, and simvastatin.

In some embodiments of the invention, the pharmacological agent comprises an antibiotic. According to the invention, suitable antibiotics include, without limitation, aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

In some embodiments of the invention, the pharmacological agent comprises a growth factor. According to the invention, suitable growth factors include, without limitation, platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-α), and placental growth factor (PLGF).

In some embodiments of the invention, the pharmacological agent comprises a steroid. According to the invention, suitable steroids include, without limitation, andranes (e.g., testosterone), cholestanes, cholic acids, corticosteroids (e.g., dexamethasone), estraenes (e.g., estradiol) and pregnanes (e.g., progesterone).

In some embodiments of the invention, the pharmacological agent comprises an anti-inflammatory.

In some embodiments of the invention, the pharmacological compositions comprise extracellular matrix (ECM) compositions that include at least one ECM material.

According to the invention, the ECM material can be derived from various mammalian tissue sources, including, without limitation, stomach tissue (e.g., stomach submucosa (SS)), small intestinal tissue (e.g., small intestinal submucosa (SIS)), large intestinal tissue, bladder tissue (e.g., urinary bladder submucosa (UBS)), liver tissue (e.g., liver basement membrane (LBM)), heart tissue (e.g., pericardium), lung tissue, kidney tissue, pancreatic tissue, prostate tissue, mesothelial tissue, fetal tissue, a placenta, a ureter, veins, arteries, tissue surrounding the roots of developing teeth, and tissue surrounding growing bone.

In some embodiments of the invention, the ECM compositions include at least one of the aforementioned pharmacological agents.

Thus, in some embodiments of the invention, the ECM compositions include a cell.

In some embodiments of the invention, the ECM compositions include a protein.

In some embodiments of the invention, the ECM compositions include a statin.

In some embodiments of the invention, the ECM compositions include chitosan.

In some embodiments of the invention there is also provided methods for improving cardiovascular function in a subject. In one embodiment, the method comprises (i) implanting an intra-myocardial device (or system) in the subject's myocardium, transmitting a first dose of a pharmacological agent to the intra-myocardial device, and administering the first dose of the pharmacological agent to the subject for a first period of time, the first dose of pharmacological agent being sufficient to cause a measurable improvement in cardiovascular function.

As set forth in detail herein, the present invention provides superior results and numerous advantages over prior art systems and methods for treating damaged or diseased cardiovascular tissue. One significant advantage of the present invention is that relatively small quantities of a pharmacological agent can be administered over an extended period of time to biological tissue; particularly, cardiovascular tissue. The methods of the present invention thus avoid the pitfalls associated with systemic delivery of a pharmacological agent.

A further advantage of the present invention is that it avoids problems associated with bolus injection of a pharmacological agent, such as delivery of an amount of agent to cardiovascular tissue that is too high and, which therefore, can have deleterious effects on the cardiovascular tissue.

Another advantage is that the intra-myocardial agent delivery systems and methods of the invention provide long-term delivery of pharmacological agents and compositions to cardiovascular tissue; particularly, myocardial tissue, with an even delivery rate, approximating to zero-order kinetics over a substantial period of delivery.

Another important advantage is that extended delivery of pharmacological agents and compositions to cardiovascular tissue can be achieved without the need for repeated invasive surgery, thereby reducing trauma to the patient.

Another advantage is that the intra-myocardial agent delivery devices and systems of the invention enhance the structural integrity of the cardiovascular structure; particularly, the myocardium when disposed therein.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a depiction of a normal heart;

FIG. 2 is an illustration of a heart having an ischemic infracted region;

FIG. 3 is a top plan view of one embodiment of an intra-myocardial agent delivery device, in accordance with the invention;

FIG. 4 is a side plan view of the intra-myocardial agent delivery device shown in FIG. 3, in accordance with the invention;

FIG. 5 is a partial side plan view of an agent delivery tube having a plurality of perforations, in accordance with the invention;

FIG. 6 is an illustration of the placement of an intra-myocardial agent delivery device in a myocardium, in accordance with one embodiment of the invention; and

FIG. 7 is a further illustration of the placement of an intra-myocardial agent delivery device in a myocardium, in accordance with one embodiment of the invention.

### DETAILED DESCRIPTION OF THE PREFERRED EMBODIMENT

Before describing the present invention in detail, it is to be understood that this invention is not limited to particularly exemplified apparatus, systems, compositions or methods as such may, of course, vary. Thus, although a number of systems, agents, compositions and methods similar or equivalent to those described herein can be used in the practice of the present invention, the preferred systems, compositions and methods are described herein.

It is also to be understood that, although the devices, systems, pharmacological agents and compositions, and methods of the invention are illustrated and described in connection with administration (or delivery) of pharmacological agents and compositions to cardiovascular tissue, the systems, compositions and methods of the invention are not limited to such delivery. According to the invention, the systems and methods of the invention can be employed to administer pharmacological agents and compositions to numerous additional biological tissue, including, without limitation, gastrointestinal and respiratory organ tissue.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an anti-inflammatory” includes two or more such agents and the like.

### DEFINITIONS

The term “cardiovascular tissue damage”, as used herein, means and includes any area of abnormal tissue in the cardiovascular system or heart caused by a disease, disorder, injury or damage, including damage to the epicardium, endocardium and/or myocardium. Non-limiting examples of causes of cardiovascular tissue damage include acute or chronic stress (systemic hypertension, pulmonary hypertension, valve dysfunction, etc.), coronary artery disease, ischemia or infarction, inflammatory disease and cardiomyopathies.

As is well known in the art, cardiovascular tissue damage most often involves damage or injury to the myocardium and, therefore, for the purposes of this disclosure, myocardial damage or injury is equivalent to cardiovascular tissue damage.

The term “damaged tissue”, as used herein, means and includes biological tissue; particularly, cardiovascular tissue damaged or injured by trauma, ischemic tissue, infarcted tissue or tissue damaged by any means which results in interruption of normal blood flow to the tissue.

The terms “prevent” and “preventing” are used interchangeably herein, and mean and include reducing the frequency or severity of a disease, condition or disorder. The term does not require an absolute preclusion of the disease, condition or disorder. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition or disorder. The terms include “active treatment”, i.e. treatment directed specifically toward the improvement of a disease, pathological condition or disorder, and “causal treatment”, i.e. treatment directed toward removal of the cause of the associated disease, pathological condition or disorder.

The terms “treat” and “treatment” further include “palliative treatment”, i.e. treatment designed for the relief of symptoms rather than the curing of the disease, pathological condition or disorder, “preventative treatment”, i.e. treatment directed to minimizing or partially or completely inhibiting the development of the associated disease, pathological condition or disorder, and “supportive treatment”, i.e. treatment employed to supplement another specific therapy directed toward the improvement of the associated disease, pathological condition or disorder.

The term “chamber remodeling”, as used herein, means and includes a series of events (which may include changes in gene expression, molecular, cellular and interstitial changes) that result in changes in size, shape and function of cardiac tissue following stress or injury. As is well known in the art, remodeling can occur after a myocardial infarction, pressure overload (e.g., aortic stenosis, hypertension), volume overload (e.g., valvular regurgitation), inflammatory heart disease (e.g., myocarditis), or in idiopathic cases (e.g., idiopathic dilated cardiomyopathy).

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “extracellular matrix”, “extracellular matrix material” and “ECM material” are used interchangeably herein, and mean a collagen-rich substance that is found in between cells in animal tissue and serves as a structural element in tissues. It typically comprises a complex mixture of polysaccharides and proteins secreted by cells. The extracellular matrix can be isolated and treated in a variety of ways. Extracellular matrix material (ECM) can be isolated from small intestine submucosa, stomach submucosa, urinary bladder submucosa, tissue mucosa, dura mater, liver basement membrane, pericardium or other tissues. Following isolation and treatment, it is commonly referred to as extracellular matrix or ECM material.

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, Anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

According to the invention, the terms “pharmacological agent”, “active agent”, “drug” and “active agent formulation” can further include, without limitation, the following growth factors: platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor alpha (TNA-α), and placental growth factor (PLGF).

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenyloin and mexiletine; (Class Ic) flecamide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocalne, ropivacaine, and trimecaine. Local anesthetics can further include combinations of the above from either amides or esters.

The terms “pharmacological agent”, “active agent”, “drug” and “active agent formulation” further include one or more classes of cytotoxic anti-neoplastic agents or chemotherapy agents, including, without limitation, alkylating agents, cisplatin, carboplatin, oxaliplatin, mechlorethamine, cyclophosphamide, chlorambucil, and ifosfamide. Chemotherapy agents can also include, without limitation, antimetabolites, such as purine analogues, pyrimidine analogues, and antifolates. Chemotherapy drugs can also include, without limitation, plant alkaloids, such as vincristine, vinblastine, vinorelbine, vindesine, podophyllotoxin, etoposide, teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomyocin, bleomycin, plicamycin, mytomycin and anthracyclines, such as doxorubicin, daunorubicin, valrubicin, idarubicin, epirubicin, and antibody treatments, such as abciximab, adamlimumab, alamtuzumab, basiliximab, belimumab, bevacizumab, brentuximab vedotin, canakinumab, cetuximab, certolizumab pego, daclizumab, denosumab, eculizumab, efalizumab, gemtuzumab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab and trastuzumab.

The terms “pharmacological agent”, “active agent”, “drug” and “active agent formulation” further include an “anti-inflammatory” and “anti-inflammatory agent”, which are used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

The terms “pharmacological agent”, “active agent”, “drug” and “active agent formulation” thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, morniflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The terms “pharmacological agent”, “active agent”, “drug” and “active agent formulation” further include “cells” and “stem cells”, which are also used interchangeably herein, and mean and include an organism that has the potential to induce modulating proliferation, and/or growth and/or regeneration of tissue.

The terms “pharmacological agent”, “active agent”, “drug” and “active agent formulation” thus include, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

According to the invention, the terms “pharmacological agent”, “active agent”, “drug” and “active agent formulation” further include the following active agents (referred to interchangeably herein as a “protein”, “peptide” and “polypeptide”): collagen (types 1-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

The terms “active agent formulation”, “pharmacological agent formulation” and “agent formulation”, are also used interchangeably herein, and mean and include an active agent optionally in combination with one or more pharmaceutically acceptable carriers and/or additional inert ingredients. According to the invention, the formulations can be either in solution or in suspension in the carrier.

The term “pharmacological composition”, as used herein, means and includes a composition comprising a “pharmacological agent” and/or a “pharmacological agent formulation” and/or any additional agent or component identified herein.

The term “pharmacological composition”, as used herein, further means and includes an extracellular matrix (ECM) composition having at least one ECM material and, in some embodiments, an additional biologically active agent, e.g., a pharmacological agent.

The term “therapeutically effective”, as used herein, means that the amount of the “pharmacological composition” and/or “pharmacological agent” and/or “active agent formulation” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “delivery” and “administration” are used interchangeably herein, and mean and include providing a “pharmacological composition” or “pharmacological agent” or “active agent formulation” to a treatment site, e.g., damaged tissue.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As will readily be appreciated by one having ordinary skill in the art, the present invention substantially reduces or eliminates the disadvantages and drawbacks associated with prior art methods of treating damaged or diseased biological tissue; particularly, cardiovascular tissue.

In overview, the present disclosure is directed to intra-myocardial agent delivery devices, systems and methods for site specific delivery of pharmacological agents and/or compositions to damaged and diseased cardiovascular tissue; particularly, myocardial tissue, and means for implanting and using the delivery systems to enable delivery of pharmacological agents and compositions to cardiovascular tissue.

In some embodiments of the invention, the intra-myocardial agent delivery devices also comprise myocardium reinforcing members that enhance the structural integrity of the myocardium.

Referring now to FIG. 1 there is shown a depiction of a normal heart 100. The heart wall 102 consists of an inner layer of simple squamous epithelium, referred to as the endocardium. The endocardium overlays the myocardium (a variably thick heart muscle) and is enveloped within a multi-layer tissue structure referred to as the pericardium. The innermost layer of the pericardium, referred to as the visceral pericardium or epicardium, covers the myocardium. An outermost layer of the pericardium, referred to as the fibrous pericardium, attaches the parietal pericardium to the sternum, the great vessels and the diaphragm.

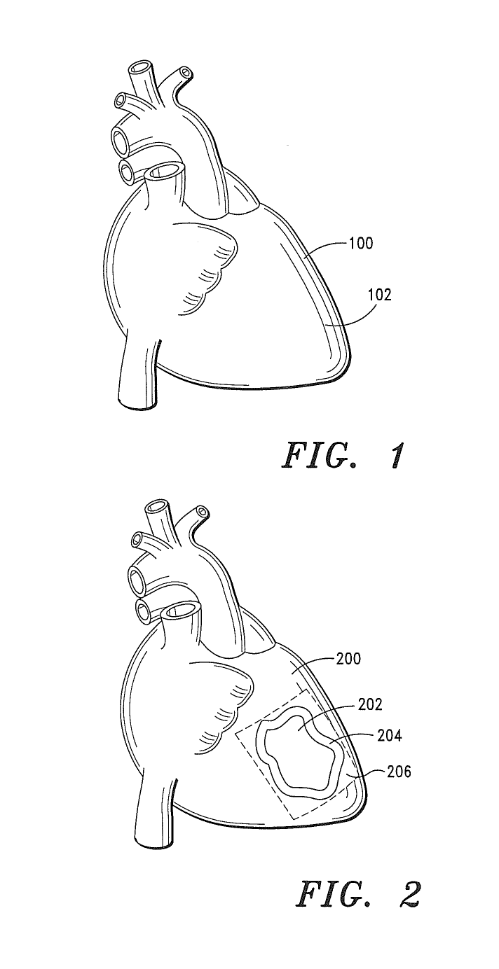

Referring now to FIG. 2, there is shown a depiction of a heart 200 having an ischemic infracted region 202, and a peri-infarcted region 204 that is surrounded by healthy non-ischemic myocardium tissue 206.

As indicated above, a myocardial infarction, i.e. irreversible myocardial injury resulting in necrosis of a significant portion of myocardium, can result in an acute depression in ventricular function and expansion of the infarcted tissue under stress. This triggers a cascading sequence of myocellular events. In many cases, this progressive myocardial infarct expansion and remodeling leads to deterioration in ventricular function and heart failure.

When a myocardial infarction occurs, the myocardial tissue that is no longer receiving adequate blood flow dies and is replaced with scar tissue. The infarct tissue cannot contract during systole, and can actually undergo lengthening in systole and, thereby, depression in ventricular function. The abnormal motion of the infarct tissue can also cause delayed or abnormal conduction of electrical activity to the still surviving peri-infarct tissue (tissue at the junction between the normal tissue and the infarcted tissue) and additionally places extra structural stress on the peri-infarct tissue.

In addition to immediate hemodynamic effects, the infarct tissue undergoes three major processes: infarct expansion, infarct extension, and chamber remodeling. These factors individually and in combination contribute to the eventual dysfunction observed in the cardiac tissue remote from the site of the infarction.

As indicated above, the noted effects of a myocardial infarction can be ameliorated or eliminated by direct administration of one or more pharmacological agents and/or compositions of the invention to the infarct tissue (or a region proximate thereto) via an intra-myocardial delivery device of the invention. As also indicated herein, the intra-myocardial delivery devices of the invention also enhance the structural integrity of the myocardium, i.e. myocardium reinforcing members.

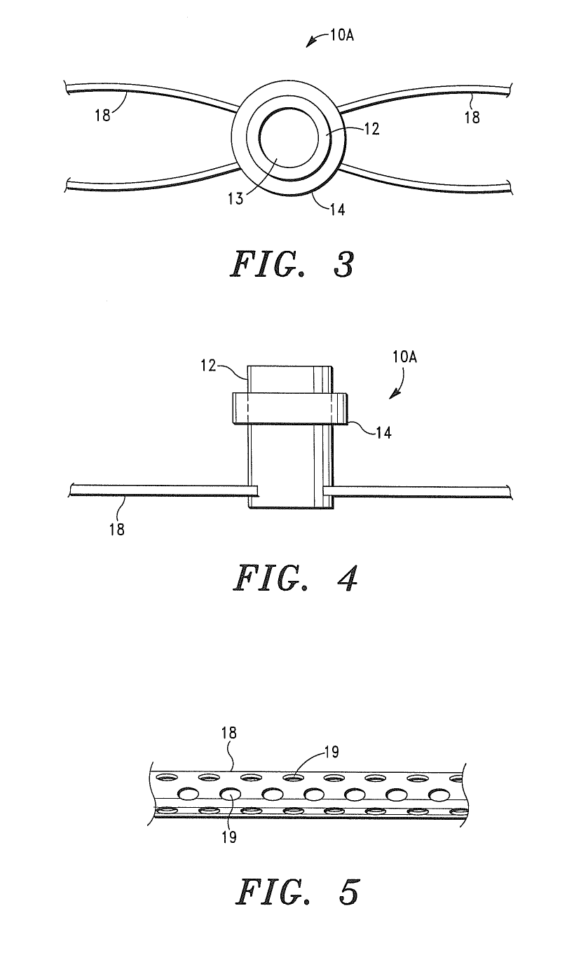

Referring now to FIG. 3-5, there is shown one embodiment of an intra-myocardial agent delivery device of the invention. As illustrated in FIGS. 3 and 4, the intra-myocardial agent delivery device 10A includes a central tube 12 with a raised shoulder region 14, and at least one, more preferably, a plurality of agent delivery tubes 18 that are in fluid communication with the central tube 12.

According to the invention, the intra-myocardial agent delivery devices of the invention can include any number of agent delivery tubes 18 to, for example, provide the desired degree of reinforcement for the myocardium. In the embodiment illustrated in FIGS. 3 and 4, the intra-myocardial agent delivery device 10A includes four (4) agent delivery tubes 18.

Further, the length of each tube 18 can also be tailored for a specific deployment. Moreover, the length of each delivery tube 18 can be the same or different.

As illustrated in FIG. 3, the central tube 12 includes a central lumen 13 that extends through the central tube 12. Each agent delivery tube 18 also includes a central lumen that is configured to facilitate the transfer of a pharmacological agent therethrough.

Referring now to FIG. 5, in a preferred embodiment of the invention, at least one, more preferably, each agent delivery tube 18 has at least one, more preferably, a plurality of laterally positioned perforations or lumens 19 that facilitate delivery of pharmacological agents from the delivery tubes 18 to adjacent biological tissue.

In some embodiments, the end of each delivery tube 18 is open. In some embodiments, the end of each delivery tube 18 is closed, whereby the pharmacological agent is delivered via the perforations or lumens 19.

According to the invention, the intra-myocardial agent delivery systems of the invention further include a pump or other agent delivery means that is designed and configured to transfer pharmacological agents to the intra-myocardial agent delivery devices, i.e. into the central tube 12 of the devices. In some embodiments of the invention, the systems include a pump having an agent dispersal line that is configured to engage to center tube 12.

According to the invention, the pump can also be implanted subcutaneously, for example, in the chest area, under the arm, etc.

According to the invention, the central tube 12 and agent delivery tubes 18 can comprise various biocompatible materials, including, without limitation, various polymeric materials, such as PEEK, polyethylene (PE), polypropylene (PE), polyvinyl chloride (PVC) and like materials.

In some embodiments of the invention, the central tube 12 and agent delivery tubes 18 comprise a biocompatible metal. Suitable biocompatible metals include, without limitation, stainless steel, titanium, tantalum, and shape-memory alloys, including, without limitation, Nitinol®.

In some embodiments of the invention, the central tube 12 and agent delivery tubes 18 comprise a biodegradable metal. Suitable biodegradable metals include, without limitation, magnesium and iron-based alloys (Mg—Al, Mg—Ca, Fe—Mn, etc.).

Other suitable biodegradable materials include polylactide (PLA), polyglycolide (PGA), poly-L-lactide (PLLA) and like materials.

In one embodiment of the invention, the central tube 12 and agent delivery tubes 18 comprise Nitinol®. In another embodiment, the agent delivery tubes 18 comprise Nitinol®.

In the noted embodiment(s), the agent delivery tubes 18 are initially formed in a desired post-deployment configuration or shape, i.e. a shape that conforms to the cardiovascular structure, e.g. myocardium, and subsequently heat-treated at a first temperature (i.e. shape set heat treatment).

The agent delivery tubes 18 are then deformed or formed in a pre-deployment configuration or shape for deployment into the cardiovascular tissue.

After the intra-myocardial agent delivery device is placed at a desired position within the target organ or structure, e.g., myocardium, the agent delivery tubes 18 transition to an austenitic phase (i.e. the temperature of the agent delivery tubes 18 reach and exceed the Nitinol® transition temperature by virtue of the body temperature) and recover (or return to) their original post-deployment configuration, whereby the agent delivery tubes 18 reinforce the structure of the target organ or structure.

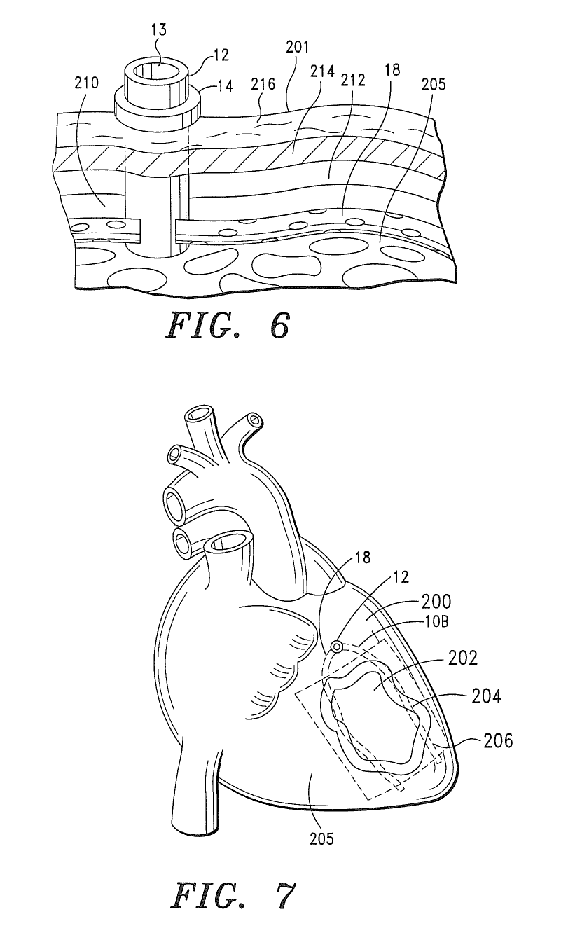

Referring now to FIGS. 6 and 7, there is shown another embodiment of an intra-myocardial agent delivery device (denoted “10B”) deployed in a myocardium 205. As illustrated in FIG. 7, the intra-myocardial agent delivery device 10B, in this instance, includes two (2) agent delivery tubes 18.

According to one embodiment of the invention, the central tube 12 and agent delivery tubes 18 are positioned in the myocardium 205; the agent delivery tubes 18 extending beneath the epicardium (visceral pericardium) 210 for a distance through myocardium tissue 206 to the infracted region 202 and/or a peri-infarcted region 204.

As illustrated in FIG. 6, in a preferred embodiment, the central tube 12 extends up through the pericardial cavity 212, the parietal pericardium 214, and the outer fibrous layer 216 of the myocardium 205.

According to the invention, the central tube 12 can then be secured to the skin, or to a pump or reservoir that delivers the pharmacological agent subcutaneously.

According to the invention, the agent delivery tubes 18 can be disposed in cardiovascular tissue, e.g. myocardium tissue, at any desired and, of course, safe depth. The agent delivery tubes 18 can also be disposed proximate or on the surface of the cardiovascular tissue.

According to the invention, the intra-myocardial agent delivery devices of the invention can be disposed on or in a myocardium (or other biological organ) via various minimally-invasive conventional procedures. In some embodiments, the intra-myocardial agent delivery devices of the invention can be disposed in a myocardium by direct or thoracoscopic visualization. In some embodiments, the leading ends of the agent delivery tubes 18 are swedged onto a needle or fed through a hollow needle slightly larger than the agent delivery tube 18 that is fed though the myocardium 205.

According to the invention, desired positioning of the central tube 12 and the agent delivery tubes 18 within the myocardium 205 (or other heart region) can be determined by various conventional means. In one embodiment, aspiration can be used to determine if the central tube 12 or the agent delivery tubes 18 have fully punctured the heart wall 201 and entered into the ventricle (or atrium). If the blood enters one or more of the agent delivery tubes 18 and exits briskly out through the central tube 12, then it can be assumed that one or more of the agent delivery tubes 18 (or the central tube 12) have punctured into the ventricle.

In another embodiment, CO₂ can be infused into the pharmacological agent and the heart can be monitored via echocardiogram. Contrast CO₂ is commonly used to test for intra-atrial features and connections.

After it has been determined that the central tube 12 and agent delivery tubes 18 have been properly placed, a pharmacological agent can be administered to desired tissues over a period of time. The intra-myocardial agent delivery devices and systems of the invention also afford the ability to administer different pharmacological agents as well, whereas an implant that does not retain connectivity can only deliver its contents.

In one exemplary embodiment, wherein the intra-myocardial agent delivery system comprises a biodegradable material, such as magnesium or PLA, pharmacological agents can be delivered over a period of time and at the conclusion of treatment, there is no need for additional surgeries in order to retrieve the implanted device.

As indicated above, in a preferred embodiment, the intra-myocardial agent delivery device and systems of the invention are designed and configured to delivery at least one pharmacological agent or composition to a target tissue site, e.g. infarct region, which can comprise any of the aforementioned pharmacological agents, including, without limitation, antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

Suitable pharmacological agents and/or compositions thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, Anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

In some embodiments of the invention, the pharmacological agent comprises a Class I, II, III or IV anti-arrhythmic agent.

In some embodiments of the invention, the pharmacological agent comprises an angiogenic factor, growth factor, antihypertensive agent, inotropic agent, antiatherogenic agent, beta-blocker, sympathomimetic agent, phosphodiesterase inhibitor, diuretic, vasodilator, thrombolytic agent, cardiac glycoside, and antineoplastic agent.

In some embodiments of the invention, the pharmacological agent comprises an anti-inflammatory agent. According to the invention, suitable anti-inflammatory agents include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, morniflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. Several significant properties and beneficial actions resulting therefrom are discussed in detail below. Additional properties and beneficial actions are set forth in Co-Pending application Ser. No. 13/373,569; which is incorporated by reference herein in its entirety.

### Anti-Inflammatory Properties/Actions

Statins have numerous favorable effects on vascular wall cells and the cardiovascular system. One specific example is that statins facilitate the reduction of the G-Protein-Coupled Receptor, thromboxane A2 (TXA₂), which lowers the platelet activation and aggregation, and augmentation of adhesion molecules and chemokines.

Statins further impact vascular wall cells and the cardiovascular system by blocking ras homilog gene family, member A (RhoA) activation. Blocking RhoA activation further impacts numerous systems, such as macrophage growth, tissue plasminogen activators (t-PA), plasminogen activator inhibitor type 1 (PAI-1), smooth muscle cell (SMC) proliferation, nitric oxide (NO) production, endothelins, and angiotensin receptors.

Macrophage growth reduced by blocking RhoA activation results in the reduction of matrix metalloprotinases (MMPs) and tissue factors (TF). Lowered MMPs also results in a lowered presence of thrombi, as the MMPs attach to ECM present in thrombi or damaged ECM at wound sites.

### Fibrinolysis Properties/Actions

Blocking RhoA activation also affects the presence of tissue plasminogen activators (t-PA) and plasminogen activator inhibitor type 1 (PAI-1), which is the principal inhibitor of fibrinolysis. With t-PA presence raised and PAI-1 diminished from the blocking of RhoA activation induced by statins, a reduced thrombotic effect is realized due to reduced opportunity for fibrin to form the polymeric mesh of a hemostatic plug.

### NO Regulation Properties/Actions

Blocking RhoA activation also affects the presence of Nitric Oxide (NO) in the cardiovascular system. NO contributes to vessel homeostasis by inhibiting vascular smooth muscle contraction and growth, platelet aggregation, and leukocyte adhesion to the endothelium.

### RhoA Activation Blocking Properties/Actions

The administration of statins can also enhance the presence of endothelins and angiotensin receptors. Endothelins and angiotensin receptors can also be affected by the subsequent blocking of RhoA activation associated with statin administration.

There are three isoforms of endothelins; ET-1, ET-2, and ET-3, with ET-1 being the isoform primarily affected by statins and RhoA activation blocking. Secretion of ET-1 from the endothelium signals vasoconstriction and influences local cellular growth and survival.

Angiotensin receptors are protein coupled receptors that are responsible for the signal transduction of the vasoconstricting stimulus of the main effector hormone angiotensin II. Angiotensin Receptor II Type I (AT-1) is the angiotensin receptor primarily affected by statin administration and RhoA activation blocking. AT-1 mediates vasocontraction, cardiac hypertrophy, vascular smooth muscle cell proliferation, inter alia.

### C-Reactive Protein Reduction Properties/Actions

C-Reactive Proteins (CRP) are also reduced by statins. CRPs are found in the blood; the levels of which deviate in response to differing levels of inflammation.

### Adhesion Molecule Reduction Properties/Actions

Statins also reduce the presence of adhesion molecules on the endothelium. Adhesion molecules are proteins that are located on the cell surface and are involved with inflammation and thrombin formation in vascular endothelial cells.

### Rac-1 Reduction Properties/Actions

The expression of Rac-1 is also reduced by statins. Rac-1 is a protein found in human cells, which plays a central role in endothelial cell migration, tubulogenesis, adhesion, and permeability. The decrease in the presence of Rac-1 also results in the decrease of reactive oxygen species (ROS).

In various aspects, the pharmacological agents (and compositions, discussed below) can be, and, preferably are, delivered at a low dose rate, e.g., up to about 0.01 microgram/hr, 0.10 microgram/hr, 0.25 microgram/hr, 1 microgram/hr, or 5, 10, 25, 50, 75, 100, 150, or generally up to about 200 microgram/hr. Specific ranges of amount of a pharmacological agent delivered to target tissue will, of course, vary depending upon numerous factors, for example, the potency.

In one exemplary embodiment, a pharmacological agent is delivered at a low volume rate e.g., a volume rate of from about 0.01 microliters/day to about 2 ml/day. Delivery of a formulation can be substantially continuous or pulsate, and can be for a pre-selected administration period ranging from several hours to years.

In some embodiments of the invention, the pharmacological compositions comprise extracellular matrix (ECM) compositions that include at least one extracellular matrix (hereinafter “ECM material”) and, optionally, one or more of the above referenced agents, e.g., an antibiotic or a statin. The ECM compositions can further include a biologically active agent, such as a cell, protein or chitosan, which are discussed below.

According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety.

According to the invention, the mammalian tissue sources can include, without limitation, stomach tissue (e.g., stomach submucosa (SS)), small intestinal tissue (e.g., small intestinal submucosa (SIS)), large intestinal tissue, bladder tissue (e.g., urinary bladder submucosa (UBS)), liver tissue (e.g., liver basement membrane (LBM)), heart tissue (e.g., pericardium), lung tissue, kidney tissue, pancreatic tissue, prostate tissue, mesothelial tissue, fetal tissue, a placenta, a ureter, veins, arteries, tissue surrounding the roots of developing teeth, and tissue surrounding growing bone.

According to the invention, the ECM compositions can comprise any known ECM component or material, including, for example and without limitation, mucosal layers and components, submucosal layers and components, muscularis layers and components, dermis, and/or basement membrane layers and components.

In some embodiments of the invention, the ECM compositions comprise sterilized acellular ECM compositions that are preferably formed by contemporaneously sterilizing and decellularizing an isolated ECM material.

Suitable sterilized acellular ECM compositions and methods for making same are set forth in Co-Pending application Ser. Nos. 13/480,140, 12/707,427, 13/480,205, and 1/747,028; which are incorporated by reference herein in their entirety.

In addition to decellularizing the ECM material, as described herein, the rapid depressurization of the ECM material can also be employed to incorporate desired sterilants and selective biologically active agents into the ECM material.

According to the invention, the ECM material can be formed into a particulate and fluidized, as described in U.S. Pat. Nos. 5,275,826, 6,579,538 and 6,933,326, to form an ECM composition of the invention.

According to the invention, various conventional means can be employed to form a particulate ECM material. In some embodiments, the ECM material is formed into a sheet, fluidized (or hydrated), if necessary, frozen and ground.

In some embodiments of the invention, the ground ECM material is subsequently filtered to achieve a desired particulate size. Thus, in some embodiments, the ECM material has a particulate size no greater than 2000 microns. In some embodiments, the ECM material preferably has a particulate size no greater than 500 microns. In a preferred embodiment, the ECM material has a particulate size in the range of about 20 microns to about 300 microns.

According to the invention, fluidized or emulsified compositions (the liquid or semi-solid forms) can comprise various certain concentrations of ECM material. In some embodiments of the invention, the concentration of the ECM material is greater than about 5%, more preferably, greater than about 20%, even more preferably, greater than about 70%.

According to the invention, the particulate ECM material can be fluidized or hydrated by various conventional buffer materials. Suitable buffer materials include, without limitation, water and saline.

According to the invention, the ECM compositions of the invention can also comprise ECM material from two or more mammalian sources. Thus, for example, the composition can comprise ECM material combinations from such sources as, for example, but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart ECM material, dermal matrix, and, in general, ECM material from any mammalian fetal tissue. The ECM material sources can also comprise different mammalian animals or an entirely different species of mammals.

According to the invention, any one of the noted tissue sources can provide material that can be formulated (or processed) into a desired form (liquid, semi-solid or solid form) for use in an ECM composition of the invention.

According to the invention, the liquid or semi-solid components of the ECM compositions (i.e. liquids, gels, emulsions or pastes) can comprise various concentrations. Preferably, the concentration of the liquid or semi-solid components of the ECM compositions is in the range of about 0.001 mg/ml to about 200 mg/ml. Suitable concentration ranges thus include, without limitation: about 5 mg/ml to about 150 mg/ml, about 10 mg/ml to about 125 mg/ml, about 25 mg/ml to about 100 mg/ml, about 20 mg/ml to about 75 mg/ml, about 25 mg/ml to about 60 mg/ml, about 30 mg/ml to about 50 mg/ml, and about 35 mg/ml to about 45 mg/ml and about 40 mg/ml. to about 42 mg/ml.

The noted concentration ranges are, however, merely exemplary and not intended to be exhaustive or limiting. It is understood that any value within any of the listed ranges is deemed a reasonable and useful value for a concentration of a liquid or semi-solid component of an ECM composition.

According to the invention, the dry particulate or reconstituted particulate that forms a gel emulsion or paste of the two ECM materials can also be mixed together in various proportions. For example, the particulates can comprise 50% of small intestine submucosa mixed with 50% of pancreatic basement membrane. The mixture can then similarly be fluidized by hydrating in a suitable buffer, such as saline.

According to the invention, the administration of a therapeutically effective amount of an ECM based pharmacological composition of the invention (with or without an additional pharmacological agent) to damaged or diseased tissue induces neovascularization, host tissue proliferation, bioremodeling and regeneration of new tissue.

As indicated above, in some embodiments of the invention, the ECM compositions of the invention include at least one of the aforementioned pharmacological agents.

Thus, in some embodiments of the invention, the ECM compositions include a statin.

In some embodiments of the invention, the ECM compositions include chitosan or a derivative thereof. As also set forth in detail in Co-Pending application Ser. No. 13/573,569, chitosan also exhibits numerous beneficial properties that provide several beneficial biochemical actions or activities.

According to the invention, the amount of chitosan added to a pharmacological composition of the invention is preferably less than 50 ml, more preferably, less than approximately 20 ml.

In some embodiments of the invention, the chitosan is incorporated in a polymeric network, such as disclosed in U.S. Pub. Nos. 2008/0254104 and 2009/0062849, which are incorporated herein in their entirety

In some embodiments of the invention, the ECM compositions include at least one of the aforementioned cells.

In some embodiments of the invention, the ECM compositions include at least one of the aforementioned proteins.

In some embodiments of the invention, the ECM compositions include at least one of the aforementioned growth factors.

According to the invention, the agents referenced above can comprise any form. In some embodiments of the invention, the agent(s), e.g. simvastatin and/or chitosan, comprise microcapsules that provide delayed delivery of the agent contained therein.

As indicated above, the effects of a myocardial infarction can be ameliorated or eliminated by direct administration of one or more of the above referenced pharmacological agents and/or compositions to the infarct tissue (or a region proximate thereto) via an intra-myocardial delivery device of the invention.

As also indicated above, a myocardial infarction is just one of many cardiovascular disorders that can be treated with an intra-myocardial agent delivery device of the invention. Indeed, intra-myocardial agent delivery devices of the invention can be readily employed to treat various additional cardiovascular disorders, including cardiac arrhythmia.

Cardiac arrhythmias are disorders involving the electrical impulse generating system of the heart. The disorders include premature contractions (extrasystoles) originating in abnormal foci in atria or ventricles, paroxysmal supraventricular tachycardia, atrial flutter, atrial fibrillation, ventricular fibrillation and ventricular tachycardia. More particularly, cardiac arrhythmia is a disorder of rate, rhythm or conduction of electrical impulses within the heart. It is often associated with coronary artery diseases, e.g., myocardial infarction and atherosclerotic heart disease.

Arrhythmia can eventually cause a decrease of mechanical efficiency of the heart, reducing cardiac output. As a result, arrhythmia can have life-threatening effects that require immediate intervention.

Ventricular arrhythmia is often deemed a premonitory sign and risk marker of sudden death. Ventricular tachycardia (VT) is most often associated with structural heart disease, such as ischemic heart disease and previous myocardial infarction, cardiomyopathy (dilated and hypertrophic), arrhythmogenic right ventricular dysplasia, and valvular heart disease (mitral valve prolapse).

Prognostic significance of VT mostly depends on the type and degree of structural heart disease and on global cardiac function. In patients with asymptomatic non-sustained VT and low risk of sudden death antiarrhythmics are often administered. Conversely, in high risk patients an automatic cardioverter-defibrillator is often implanted.

In the treatment of an acute attack of VT, lidocaine, amiodarone, beta-blockers, and occasionally magnesium or verapamil is often administered. In the prevention of recurrent arrhythmia and sudden death amiodarone, sotalol, mexiletin, phenyloin, and beta-blockers are often administered.

Atrial fibrillation (AF) is the most common sustained tachyarrhythmia encountered by clinicians. AF occurs in approximately 0.4% to 1.0% of the general population, and it affects more than 2 million people in the United States annually. Its prevalence increases with age, and up to 10% of the population older than 80 years has been diagnosed with AF at some point. With the projected growth of the elderly population the prevalence of AF will certainly increase.

AF occurs when the electrical impulses in the atria degenerate from their usual organized pattern into a rapid chaotic pattern. This disruption results in an irregular and often rapid heartbeat that is classically described as “irregularly irregular” and is due to the unpredictable conduction of these disordered impulses across the atrioventricular (AV) node.

AF can be classified on the basis of the frequency of episodes and the ability of an episode to convert back to sinus rhythm. One method of classification is outlined in guidelines published by the American College of Cardiology (ACC), the American Heart Association (AHA) and the European Society of Cardiology (ESC), with the collaboration of the North American Society of Pacing and Electrophysiology (NASPE). According to these guidelines, if a patient has two or more episodes, AF is considered to be recurrent.

If the AF terminates spontaneously, it is designated as paroxysmal, and if the AF is sustained, it is designated as persistent. In the latter case, termination of the arrhythmia with electrical or pharmacologic cardioversion does not change its designation. Persistent AF may present either as the first manifestation of the arrhythmia or as the culmination of recurrent episodes of paroxysmal AF. Persistent AF also includes permanent AF, which refers to long-standing (generally >1 year) AF for which cardioversion was not indicated or attempted.

AF is often associated with physiologic stresses, such as surgical procedures, pulmonary embolism, chronic lung diseases, hyperthyroidism, and alcohol ingestion. Disease states commonly associated with AF include hypertension, valvular heart disease, congestive heart failure (CHF), coronary artery disease, Wolff-Parkinson-White (WPW) syndrome, pericarditis, and cardiomyopathy. When no identifiable risk factor for AF is present, the condition is classified as lone AF.

AF can have hemodynamic consequences. It can decrease the cardiac output by as much as 20%, increase pulmonary capillary wedge pressure, and increase atrial pressures. These effects are due to tachycardia, loss of atrial contribution to left ventricular (LV) filling, increased valvular regurgitation, and the irregular ventricular response.

AF typically occurs in 10% to 65% of patients after cardiac surgery. AF thus frequently complicates cardiac surgery and can, and often does, result in extended post-operative hospitalization. However, if an anti-arrhythmic agent could be directly administered to the heart, it could prevent or diminish post-operative atrial fibrillation and therefore improve treatment, reduce hospitalization time, and reduce cost.

Various anti-arrhythmic agents have thus been administered to patients to prevent or diminish atrial fibrillation. The anti-arrhythmic agents are commonly divided into four classes according to their electro-physiological mode of action. The four classes of anti-arrhythmic agents comprise: local anesthetic effect (Class I), beta-receptor blockade (Class II), prolongation of action potential duration (Class III), and calcium antagonism (Class IV).

The Class I anti-arrhythmic agents, e.g., sodium channel depressors, usually have little or no effect on action potential duration and exert local anesthetic activity directly at cardiac cell membrane. The Class II agents, e.g., propranolol, show little or no effect on the action potential and exert their effects through competitive inhibition of beta-adrenergic receptor sites, thereby reducing sympathetic excitation of the heart.

The Class III anti-arrhythmic agents, e.g., amiodarone and bretylium, are characterized by their ability to lengthen the action potential duration, thereby preventing or ameliorating arrhythmias. The Class IV agents, e.g., calcium antagonists, are those which have an anti-arrhythmic effect due to their actions as calcium antagonists.

Thus, in some embodiments of the invention, the pharmacological agent comprises at least one of following Class I-Class V antiarrhythmic agents: (Class Ia) quinidine, procainamide and disopyramide; (Class Ib) lidocaine, phenyloin and mexiletine; (Class Ic) flecamide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

As set forth in Co-Pending application Ser. Nos. 12/707,427 and 13/480,140, which are incorporated by reference herein, Applicant has also found that the administration of one or more of the aforementioned statins also prevent or diminish AF.

### EXAMPLES

The following examples are provided to enable those skilled in the art to more clearly understand and practice the present invention. They should not be considered as limiting the scope of the invention, but merely as being illustrated as representative thereof.

### Example 1

A 50 year old male, weighing 235 lbs., presents with a myocardium infarction.

An ECM based pharmacological composition that includes a particulate SIS extracellular matrix material, 3 mg of cerivastatin, and 10 ml chitosan is prepared and charged into a delivery pump.

An intra-myocardial agent delivery device having four (4) agent delivery tubes is implanted in the subject's myocardium. The agent delivery tubes are positioned within the myocardium wherein two of the tubes are disposed proximate the infarct region.

The pump is connected to the inlet or central tube of the device and the pharmacological composition is delivered into and through device, and into the myocardium tissue at a rate of approximately 0.25 micrograms/hr.

Within a period of 10-14 days, the effects of the myocardial infarction are ameliorated, and neovascularization, host tissue proliferation, bioremodeling and regeneration of new tissue are evident.

As will readily be appreciated by one having ordinary skill in the art, the present invention provides numerous advantages compared to prior art methods and systems for treating damaged cardiac tissue. Among the advantages are the following:
The provision of intra-myocardial agent delivery systems and methods that provide effective and accurate means for delivering pharmacological agents directly to cardiovascular tissue and/or regions proximate thereto to treat damaged or diseased cardiovascular tissue, such as infarct tissue.
The provision of intra-myocardial agent delivery systems and methods that provide effective and accurate means for delivering relatively small and controlled quantities of a pharmacological agent to biological tissue; particularly, cardiovascular tissue, over an extended period of time.
The provision of intra-myocardial agent delivery systems and methods that eliminate problems associated with bolus injection of a pharmacological agent, such as delivery of an amount of agent to cardiovascular tissue that is too high and can have deleterious effects on the cardiovascular tissue.
The provision of intra-myocardial agent delivery systems and methods that can provide long-term delivery of pharmacological agents and compositions to cardiovascular tissue; particularly, myocardial tissue, with an even delivery rate, approximating to zero-order kinetics over a substantial period of delivery.
The provision of intra-myocardial agent delivery systems and methods that can provide extended delivery of pharmacological agents and compositions to cardiovascular tissue without the need for repeated invasive surgery, thereby reducing trauma to the patient.
The provision of intra-myocardial agent delivery systems and methods that can be employed to reinforce the myocardium or a refract region thereof and do not require an open chest procedure for placement proximate the myocardium.
The provision of intra-myocardial agent delivery devices and systems that enhance the structural integrity of a cardiovascular structure; particularly, the myocardium when disposed therein.
The provision of extracellular matrix (ECM) compositions, which, when delivered to damaged biological tissue; particularly, cardiovascular tissue, induce neovascularization, host tissue proliferation, bioremodeling, and regeneration of cardiovascular tissue and associated structures with site-specific structural and functional properties.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An intra-myocardial agent delivery device, comprising:
a myocardial tissue access line that is configured to receive a first pharmacological agent therein and provide direct fluid access of said first pharmacological agent to first myocardial tissue, and at least a first agent delivery line having a wall and a central lumen therethrough, said first agent delivery line being in fluid communication with said tissue access line,
said first agent delivery line further including a plurality of dispersal lumens extending through said delivery line wall and in fluid communication with said central lumen, wherein, when said first agent delivery line is disposed in said first myocardial tissue and said first pharmacological agent is introduced into said tissue access line, said plurality of dispersal lumens allow said first pharmacological agent to be transferred from said central lumen to said first myocardial tissue.

2. The agent delivery device of claim 1, wherein said device includes a plurality of said first agent delivery lines.

3. The agent delivery device of claim 1, wherein said device includes an external pump, said pump being in fluid communication with said tissue access line and configured to introduce said first pharmacological agent into said tissue access line with a first fluid pressure.

4. The agent delivery device of claim 1, wherein said first pharmacological agent is selected from the group consisting of antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

5. The agent delivery device of claim 1, wherein said first pharmacological agent is selected from the group consisting of antihypertensive agents, inotropic agents, antiatherogenic agents, beta-blockers, sympathomimetic agents, phosphodiesterase inhibitors, diuretics, vasodilators, thrombolytic agents, cardiac glycosides, and antineoplastic agents.

6. The agent delivery device of claim 1, wherein said first pharmacological agent comprises an anti-arrhythmic agent selected from the group consisting of Class I, II, III and IV anti-arrhythmic agents.

7. The agent delivery device of claim 1, wherein said first pharmacological agent comprises a statin selected from the group consisting of atorvastatin, cerivastatin, fluvastatin, lovastatin, mevastatin, pitavastatin, pravastatin, rosuvastatin, and simvastatin.

8. The agent delivery device of claim 1, wherein said first pharmacological agent comprises an antibiotic selected from the group consisting of aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

9. The agent delivery device of claim 1, wherein said first pharmacological agent comprises a growth factor selected from the group consisting of platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor-α (TNA-α), and placental growth factor (PLGF).

10. The agent delivery device of claim 1, wherein said first pharmacological agent comprises a steroid selected from the group consisting of andranes, cholestanes, cholic acids, corticosteroids, estraenes and pregnanes.

11. The agent delivery device of claim 1, wherein said first pharmacological agent comprises an anti-inflammatory.

12. The agent delivery device of claim 1, wherein said first pharmacological agent comprises an extracellular matrix (ECM) composition, said ECM composition including an ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

13. The agent delivery device of claim 12, wherein said ECM composition includes at least one supplemental biologically active agent.

14. The agent delivery device of claim 13, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

15. The agent delivery device of claim 13, wherein said biologically active agent comprises a cell selected from the group consisting of a human embryonic stem cell, fetal cardiomyocyte, myofibroblast, mesenchymal stern cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stein cell.

16. The agent delivery device of claim 13, wherein said biologically active agent comprises a statin selected from the group consisting of atorvastatin, cerivastatin, fluvastatin, lovastatin, mevastatin, pitavastatin, pravastatin, rosuvastatin, and simvastatin.

17. The agent delivery device of claim 13, wherein said biologically active agent comprises chitosan.

18. A method of treating a cardiovascular disorder, comprising:
providing a first pharmacological agent that is effective in treating the cardiovascular disorder;
providing an agent delivery device, said agent delivery device including a tissue access line that is configured to receive said first pharmacological agent therein and at least a first agent delivery line having a wall and a central lumen therethrough, said first agent delivery line being in fluid communication with said tissue access line, said first agent delivery line further including a plurality of dispersal lumens extending through said delivery line wall and in fluid communication with said central lumen, wherein, when said first agent delivery line is disposed in first cardiovascular tissue and said first pharmacological agent is introduced into said tissue access line, said plurality of dispersal lumens allow said first pharmacological agent to be transferred from said central lumen to said first cardiovascular tissue;
disposing said agent delivery device in said first cardiovascular tissue;
introducing said first pharmacological agent into said tissue access line, wherein said first pharmacological agent is delivered to said first cardiovascular tissue.

19. The method of claim 18, wherein said agent delivery device includes a plurality of said first agent delivery lines.

20. The method of claim 18, wherein said first pharmacological agent is selected from the group consisting of antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

21. The method of claim 18, wherein said first pharmacological agent comprises an anti-arrhythmic agent selected from the group consisting of Class I, II, III and IV anti-arrhythmic agents.

22. The method of claim 18, wherein said first pharmacological agent comprises a statin selected from the group consisting of atorvastatin, cerivastatin, fluvastatin, lovastatin, mevastatin, pitavastatin, pravastatin, rosuvastatin, and simvastatin.

23. The method of claim 18, wherein said first pharmacological agent comprises a growth factor selected from the group consisting of platelet derived growth factor (PDGF), epidermal growth factor (EGF), transforming growth factor-α (TGF-α), transforming growth factor-β(TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor-α (TNA-α), and placental growth factor (PLGF).

24. The method of claim 18, wherein said first pharmacological agent comprises an extracellular matrix (ECM) composition, said ECM composition including an ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

25. The method of claim 24, wherein said ECM composition further includes a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

26. The method of claim 24, wherein said ECM composition further includes a statin selected from the group consisting of atorvastatin, cerivastatin, fluvastatin, lovastatin, mevastatin, pitavastatin, pravastatin, rosuvastatin, and simvastatin.

27. A method of treating damaged myocardial tissue in a cardiovascular structure, comprising:
providing a first pharmacological agent that is effective in treating the damaged myocardial tissue;
providing an agent delivery device, said agent delivery device including a myocardial tissue access line that is configured to receive said first pharmacological agent therein, and at least a first agent delivery line having a wall and a central lumen therethrough, said first agent delivery line being in fluid communication with said tissue access line, said agent delivery line being capable of transitioning from a pre-deployment configuration prior to being placed in first myocardial tissue to a post-deployment configuration when said agent delivery line is placed in said first myocardial tissue, said agent delivery line being formed in a post-deployment configuration, said post-deployment configuration conforming to a shape of said cardiovascular structure,
said first agent delivery line further including a plurality of dispersal lumens extending through said delivery line wall and in fluid communication with said central lumen, wherein, when said first agent delivery line is disposed in said first myocardial tissue and said first pharmacological agent is introduced into said tissue access line, said plurality of dispersal lumens allow said first pharmacological agent to be transferred from said central lumen to said first myocardial tissue;
forming said first agent delivery line in said pre-deployment configuration;
disposing said agent delivery device in said first myocardial tissue, wherein said agent delivery line is disposed proximate said damaged myocardial tissue, and wherein said agent delivery line transitions from said pre-deployment configuration to said post-deployment configuration, whereby said agent delivery line reinforces said cardiovascular structure; and
introducing said first pharmacological agent into said tissue access line, wherein said first pharmacological agent is delivered proximate said damaged myocardial tissue.

28. The method of claim 27, wherein said agent delivery line comprises Nitinol®.

29. The method of claim 27, wherein said agent delivery device includes a plurality of said first agent delivery lines.

30. The method of claim 27, wherein said first pharmacological agent is selected from the group consisting of antibiotics or antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

31. The method of claim 27, wherein said first pharmacological agent comprises an anti-arrhythmic agent selected from the group consisting of Class I, II, III and IV anti-arrhythmic agents.

32. The method of claim 27, wherein said first pharmacological agent comprises a statin selected from the group consisting of atorvastatin, cerivastatin, fluvastatin, lovastatin, mevastatin, pitavastatin, pravastatin, rosuvastatin, and simvastatin.

33. The method of claim 27, wherein said first pharmacological agent comprises a growth factor selected from the group consisting of platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), growth factor (IGF), nerve growth factor (NGF), platlet derived growth factor (PDGF), tumor necrosis factor-α (TNA-α), and placental growth factor (PLGF).

34. The method of claim 27, wherein said first pharmacological agent comprises an extracellular matrix (ECM) composition, said ECM composition including an ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

35. The method of claim 34, wherein said ECM composition further includes a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

36. The method of claim 34, wherein said ECM composition further includes a statin selected from the group consisting of atorvastatin, cerivastatin, fluvastatin, lovastatin, mevastatin, pitavastatin, pravastatin, rosuvastatin, and simvastatin.
